(19) United States
(12) Patent Application Publication
(10) Pub. No.: US 2005/0137540 A1
(43) Pub. Date: Jun. 23, 2005

(54) Bacteria removing wipe

(75) Inventors: Julie M. Villanueva, Decatur, GA (US); Curtis Neil Sayre, Atlanta, GA (US); Lei Huang, Duluth, GA (US); Kevin Peter McGrath, Alpharetta, GA (US); Ning Wei, Roswell, GA (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/745,266

(22) Filed: Dec. 23, 2003

(51) Int. Cl. A61F 13/15

(52) U.S. Cl. 604/360

(57) ABSTRACT

There is provided a wipe for the removal of bacteria from surfaces. The wipe has a positive charge that may be developed through the use of cationic treatments. The wipe may be dipped in an aqueous solution of a non-antimicrobial treatment having a positive charge and the excess solution squeezed out of the wipe. Treatment of the resulting coated wipe with heat at a temperature and for a time sufficient adheres the coating to the wipe. Alternatively, a non-antimicrobial, cationically charged chemical may be imbedded in the web such that it will bloom to the surface when the wipe is exposed to water. The web is desirably a pulp and synthetic fiber fabric made by coforming or hydroengling and may be a laminate including other layers. The treated web removes a substantial amount of the bacteria from a surface yet does not appreciably inhibit the growth of the bacteria. The web does not contain harsh chemicals and so is mild in its effect on the user's skin. The removal of the bacteria in contrast to killing the bacteria does not encourage it to develop immunity to the treatment.

### BACKGROUND OF THE INVENTION

The invention concerns processes and products for the removal of bacteria by wipes, without the use of harsh chemicals.

A myriad of different types of disposable wipes are commercially available in today's marketplace. These wipes may be smooth or rough and contain chemicals designed with a particular use in mind. These uses include as antiseptic wipes for wound care, floor and furniture cleaning wipes, automotive rejuvenation wipes, countertop cleaning wipes and hand cleaning wipes. The common aspect of these wipes is that they contain additives or chemicals to affect their purpose. Household kitchen countertop wipes, for example, generally contain chemicals that will kill bacteria.

As concern grows about allergic reactions to chemicals and about the increasing resistance of bacteria to common drug treatments, so has the concern and desire for a wipe that avoids harsh chemicals yet still achieves its purpose. Existing wet wipes are a wipe impregnated with a solution of chemical, for example an antimicrobial chemical. Use of the wipe helps deliver the chemicals to the contaminated surfaces. However, it is desirable that, after wiping the surface, the wipe retains the chemicals while removing the germs from the surface. A wipe that removes the bacteria but which does not leave chemicals on the surface provides the desired decontamination effect without the undesirable exposure to the antimicrobial chemicals. The instant invention successfully addresses this consumer desire.

### SUMMARY OF THE INVENTION

In response to the foregoing difficulties encountered by those of skill in the art, we have developed a wipe for the removal of bacteria from surfaces. The wipe has a positive charge that may be developed through the use of cationic treatments. The chemicals used to treat the wipe may be functionalized polymers, organic or inorganic oligomers, or particles coated with functionalized polymers, organic or inorganic oligomers. After the treatment is applied on the wipe, the resulting coated wipe may be treated with heat at a temperature and for a time sufficient to crosslink the coating and attach the coating to the substrate. The web is desirably a pulp and synthetic fiber fabric made according by coforming or hydroentangling and may be a laminate including other layers. The treated web removes a substantial amount of the bacteria from a surface yet does not appreciably inhibit the growth of the bacteria.

### BRIEF DESCRIPTION OF THE DRAWINGS

FIG. 1 shows a suitable bonding pattern as taught in U.S. Pat. No. 5,964,742.

FIG. 2 is a drawing of a wipe having a point un-bonded bond pattern according to U.S. Pat. No. 5,858,515.

FIG. 3 is a drawing of a method of treating a web.

### DETAILED DESCRIPTION OF THE INVENTION

The present invention involves the removal of bacteria from surfaces through the use of a wipe. This has become increasingly important to consumers as the number of bacteria resistant to common treatments has risen. It is also increasingly important to consumers that they not be exposed to chemicals merely for household or hand cleaning.

The instant invention is a wipe that removes bacteria from surfaces. The bacteria are removed without the use of harsh chemicals, i.e., chemicals that are caustic or cause irritation to the skin of the average person, so the consumer is not required to touch a chemical laden wipe. Since the bacteria are not exposed to chemicals that will kill them in substantial numbers, i.e., more than 20 percent, the bacteria are not prompted to develop immunities to the inventive wipes. The bacteria are simply removed from the surface through the application of physical means and electrical attraction, and the wipe properly disposed of.

The wipes that may be used as the base substrate in the practice of the invention are usually made from nonwoven webs or fabrics like spunbond, meltblown and coform materials. Bonded-carded webs and airlaid materials may also be used as may laminates of any of the commonly known nonwovens. Cellulosic materials like paper towels may also be used. Wipes may also be made from processes that introduce texture and increase loft such as by creping, by zero strain stretch bonding, point un-bonding, Z-directional orienting, and other means.

Nonwoven fabrics are generally bonded in some manner as they are produced in order to give them sufficient structural integrity to withstand the rigors of further processing into a finished product. Bonding can be accomplished in a number of ways such as hydroentanglement, needling, ultrasonic bonding, adhesive bonding, stitchbonding, through-air bonding and thermal bonding, all of which are suitable for the practice of this invention.

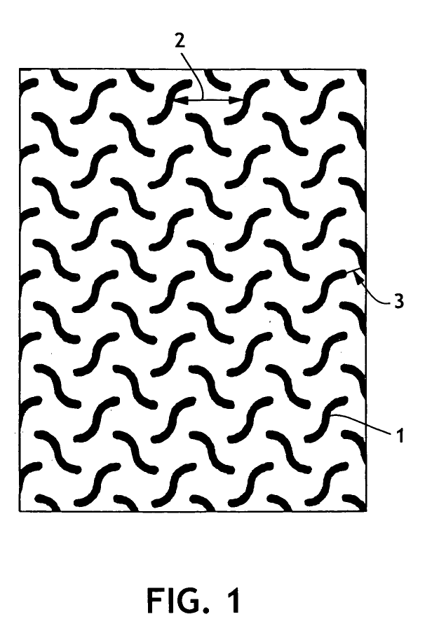

FIG. 1 shows another suitable bonding pattern for fabric yielding improved strength and abrasion resistance as taught in U.S. Pat. No. 5,964,742. The pattern elements 1 in FIG. 1 have a center spacing 2 and a minimum spacing 3 and the pattern is commonly called an S-weave pattern. This pattern has an element aspect ratio, the ratio of the length to the width, between about 2 and 20 and an unbonded fiber aspect ratio, the ratio of the center spacing to the minimum spacing, of between about 3 and about 10.

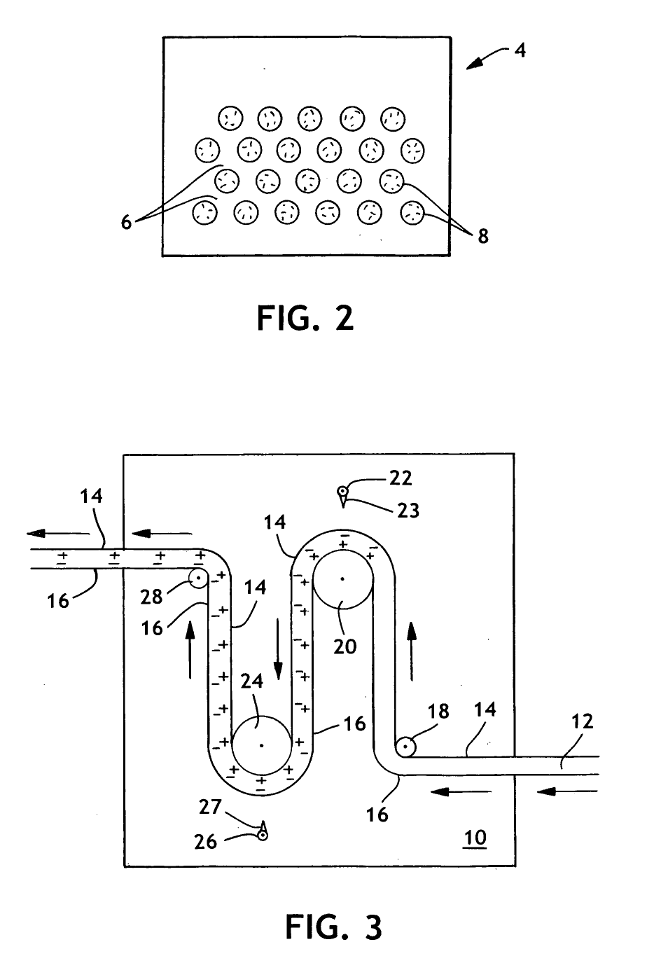

FIG. 2 shows yet another way of bonding a nonwoven web and results in the production of a point un-bonded nonwoven fabric 4 having continuous bonded area 6 surrounding and defining a plurality of discrete unbonded areas 8 according to U.S. Pat. No. 5,858,515. This fabric provides a three dimensional texture to a web.

The wipe of the invention should have a positive charge in order to attract and hold bacteria, which generally have a negative charge. Positive charges may be generated in a number of ways; a cationically charged chemical treatment may be added to the web, for example, and/or; an electret treatment may be applied to the wipe, resulting in a positive charge. Suitable chemicals include functionalized cationically charged polymers, and inorganic or organic oligomers. Nanoparticles coated with functionalized cationically charged polymers or inorganic or organic oligomers. An example of a suitable inorganic oligomer is aluminium chlorhydrol.

Chemicals useful in the generation of positive charge on a wipe include cationic polymers sold under the tradenames Kymene®, Reten®, from Hercules Inc., of Wilmington, Del., USA, Cobond® from National Starch and Chemical Company of Bridgewater, N.J., USA and Calgon polymers from Calgon Inc. of Pittsburgh, Pa., USA, and others like polyethyleneimine, high charge density polyelectrolytes like poly(methacryloxyethyl) trimethylammonium bromide poly(acrylic acid) and epichlorohydrin-functionalized polyamines. Nanoparticles like Snowtex® AK from Nissan Chemicals Inc., of Houston, Tex., USA and aluminum chlorhydrate from Reheis, Inc. of Berkeley Heights, N.J., USA, may also be used. In addition to having a positive charge, the chemicals suitable for the practice of the invention are mild in their effect on the skin, not appreciably antimicrobial in nature and do not leach substantially from the wipe.

The amount of chemical that should be added to the wipe will vary according to the amount of charge the particular chemical chosen will contribute. Generally however, the effective amount of chemical will be between about 0.01 and 10 weight percent, more desirably between 0.05 and 7 weight percent, and still more desirably between 0.1 and 5 weight percent.

The chemical treatment may be applied by methods such as traditional dip and squeeze techniques, where the wipe is dipped into the chemical treatment and excess chemical is squeezed off, or by coating, spraying, ink-jet printing, and the like.

The chemically treated wipe may be treated with heat at a temperature and for a time sufficient to crosslink the coating and adhere it to the web. The crosslinking process for functionalized cationically charged polymers involves reaction between crosslinkable functional groups (e.g., epoxy group) of the coating with either another functional group of the coating (e.g., hydroxyl group) or with a substrate functional group. For example, the substrate could be cellulose where hydroxyl groups of the fibers would intermolecularly crosslink with epoxy groups of the coating. In the case of alumina oligomers, the crosslinking process involves Al—OH groups of the oligomer and OH from either the oligomer (intramolecular crosslinking) or OH group from the substrate (intermolecular crosslinking). It's believed that the nanoparticles coated with alumina oligomer would adhere to OH-containing surfaces by crosslinking the OH group with Al—OH groups of the oligomer. The combination of time and temperature sufficient to crosslink the polymer will depend on the polymer and substrate chosen. Generally speaking however, the time will be between 1 and 60 minutes, more desirably between 5 and 45 minutes, still more desirably between 15 and 35 minutes, with a temperature between about 50 and 300° C., more desirably between about 80 and 200° C., still more desirably between about 90 and 125° C. The inventors have found, for example, that a temperature of 100° C. for about 20 to 30 minutes cures many of the polymers of interest.

Depending on the nature of the fibers, functionlized polymers (such as Kymenes® with epoxy groups) are capable of involving both intra-molecular (i.e., only within the coating layer) and inter-molecular (i.e., only with the substrate) crosslinking processes. It's believed to be likely that the crosslinking process will combine both intra-molecular and intermolecular processes if the substrate webs are functionalized. Alternatively, if the substrate is not capable of participating in the chemical crosslinking process, then only intramolecular crosslinking may occur. In either case, a durable coating is often obtained when the non-functionalized web is made wettable by pre-treating before coating. The term “adhere to the wipe” includes, therefore, instances of intramolecular crosslinking that create a “sleeve” around the fibrous substrate, as well as intermolecular crosslinking where the chemical or a carrier of the chemical (such as a Nanoparticle coated with an alumina oligomer) forms a covalent bond on the substrate, and combinations thereof. A cationically charged chemical “adheres to the wipe” if it does not leach from the wipe during the wiping process, where “not leach” from a wipe means that the concentration of the chemical in the liquid left on a wiped surface is less than the critical concentration for it to have antimicrobial property.

Alternatively, the cationically charged compound may be imbedded in the wipe by melt-extruding the fiber-forming polymer containing a desired amount of the cationically charged compound as an additive in the fibers of the web such that they will “bloom” to the surface when the web is exposed to hydrophilic solvents such as water. These melt extrudable fibers may contain a polyolefin and a cationically charged compound. The cationically charged compound also contains a chemical segment (i.e., compatibilizer) that is soluble in the polyolefin such that the salt is compatibilized with the polymer. The cationically charged chemicals may be, for example, amphiphilic quaternium ammonium salts that are compatible with hydrophobic webs, examples of which are taught by Nohr and Macdonald in U.S. Pat. No. 5,853,883, which is incorporated herein by reference. If the hydrophobic segment of the salt that is compatible with the hydrophobic polymer is relatively large (with respect to the ionic segment of the salt) such that the amount of salt that leaches out of the web is insufficient to kill bacteria, then the web would not have antimicrobial activity. The cationically charged groups will come to the surface of the predominately polymeric fibers when the web is exposed to water. Such blooming gives the webs properties similar to those of webs coated with cationically charged compounds.

Another means of inducing an electrical charge on the web is through the use of electret treatment. Electret treated webs remove bacteria by drawing the bacteria into the web by virtue of their electrical charge. Electret treatment can be carried out by a number of different techniques, generally on the web material from which the wipe will be made. One technique is described in U.S. Pat. No. 5,401,446 to Tsai et al. assigned to the University of Tennessee Research Corporation and incorporated herein by reference in its entirety. Tsai describes a process whereby a web or film is sequentially subjected to a series of electric fields such that adjacent electric fields have substantially opposite polarities with respect to each other, as shown in FIG. 3. Thus, one side 14, 16 of the web or film 12 is initially subjected to a positive charge while passing around a charging drum 20 between the drum 20 and a charging bar 22 having a point emitter 23, while the other side of the web or film is initially subjected to a negative charge while passing around a second charging drum 24 between the drum 24 and a second charging bar 26 having a point emitter 27. Then, the first side of the web or film is subjected to a negative charge and the other side of the web or film is subjected to a positive charge. Additional rollers 18, 28 help direct the web in the desired direction. Such webs are produced with a relatively high charge density without an attendant surface static electrical charge. The process may be carried out by passing the web through a plurality of dispersed non-arcing electric fields which may be varied over a range depending on the charge desired in the web. The web may be charged at a range of about 1 kVDC/cm to 12 kVDC/cm or more particularly 4 kVDC/cm to 10 kVDC/cm and still more particularly 7 kVDC/cm to about 8 kVDC/cm.

Other methods of electret treatment are known in the art such as that described in U.S. Pat. Nos. 4,215,682 to Kubik et al, 4,375,718 to Wadsworth, 4,592,815 to Nakao and 4,874,659 to Ando.

Should it be desired to both electret treat and chemically treat the wipe, the inventors recommend that the chemical treatment be performed first, the wipe allowed to dry, and then the electret treatment applied.

The inventors tested numerous substrates and chemical treatments for their bacterial removal efficiency and chemical leaching. These materials, treatments, test procedures and results are shown below.

PBS Control: This refers to sterile phosphate buffered saline (PBS) and indicates that no fabric, treatment or bacteria were present in this sample. Phosphate buffered saline (available from Gibco and Invitrogen at 10× concentration) is diluted to 1× with distilled water and sterile filtered before using.

Spunlace fabric: The spunlace process is also known as hydroentanglement. The spunlace process subjects the fiber web to fine jets of water at high pressures. When the jets strikes the web, it repositions and entangles the fibers into an interlocked “spunlace” web. The web is then dried in hot ovens. Generally speaking, spunlace webs contain no chemical binders, and they have an excellent textile-like drape and softness, good mechanical and aesthetic properties, and good absorbency and wetting. A wide range of natural and synthetic fibers can be used to make spunlace webs, including polypropylene, Rayon, PET, and nylon. Staple fibers are also used in spunlace nonwovens products. The spunlace fabric tested herein was made from 65 weight percent Rayon and 35 weight percent PET. The fabric was tested in the uncreped state as well as in the creped state where the creping was carried out in accordance with U.S. Pat. Nos. 6,197,404 and 6,150,002 which are incorporated herein in their entirety by reference thereto for all purposes. These creped materials have regions of interfilament bonding which are permanently bent out-of-plane, alternating with regions of no interfilament bonding. The non-bonded regions include a multiplicity of filament loops which terminate at bond ends in the creped interfilament bonded regions.

Fuzzy film, polyurethane: This fuzzy film is made of polyurethane (PU) foam and polyethylene (PE) film through a tack-spinning process. In tack-spinning, the PU and PE are laminated together, the PE is partially melted on a hot roller and the surface is fiberized by pulling the material away from the roller and blowing air on/through it to cool it.

Bonded Carded web: “Bonded carded web” refers to webs which are made from staple fibers which are sent through a combing or carding unit, which breaks apart and aligns the staple fibers in the machine direction to form a generally machine direction-oriented fibrous nonwoven web. Such fibers are usually purchased in bales which are placed in a picker which separates the fibers prior to the carding unit. Once the web is formed, it then is bonded by one or more of several known bonding methods. One such bonding method is powder bonding, wherein a powdered adhesive is distributed through the web and then activated, usually by heating the web and adhesive with hot air. Another suitable bonding method is pattern bonding, wherein heated calender rolls or ultrasonic bonding equipment are used to bond the fibers together, usually in a localized bond pattern, though the web can be bonded across its entire surface if so desired. Another suitable and well-known bonding method, particularly when using bicomponent staple fibers, is through-air bonding.

Textured coform laminate (TCL): This material was an elastic laminate having outer layers on either side of a core. The outer layers had a basis weight of 35 grams per square meter (gsm) each and made according to the coform process, from a blend of 60 weight percent CF405 fiberized southern softwood pulp from Weyerhaeuser Corp. and 40 weight percent PF-105 polypropylene meltblown fibers from Basell Polyolefins Company N.V. of Hoofddorp, the Netherlands. The core was 30 gsm in basis weight and made of filaments and nonwoven fabric. The filaments comprised 70 weight percent of the core and were made from Affinity® metallocene-based polyethylene from the Dow Chemical Company of Midland, Mich., USA. The nonwoven fabric was made according to the meltblown process from 80 weight percent Affinity® polyethylene, 15 weight percent Regalrez® 1126 hydrocarbon resin from Eastman Chemical Company of Kingsport, Tenn., USA and 5 weight percent DNDB 1077 linear low density polyethylene from the Dow Chemical Company.

In the meltblown process, fibers are formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. The meltblown fibers are then carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface.

In the coform process, at least one meltblown diehead is arranged near a chute through which other materials are added to a meltblown web while it is forming. Such other materials may be natural fibers, superabsorbent particles, natural polymers (for example, rayon) and/or synthetic polymers (for example, polypropylene or polyester) fibers, for example, where the fibers may be of staple length. Coform processes are shown in commonly assigned U.S. Pat. Nos. 4,818,464 to Lau and 4,100,324 to Anderson et al. Webs produced by the coform process are generally referred to as coform materials. Natural fibers include wool, cotton, flax, hemp and wood pulp. Wood pulps include standard softwood fluffing grade such as CR-1654 (US Alliance Pulp Mills, Coosa, Ala.). Pulp may be modified in order to enhance the inherent characteristics of the fibers and their processability. Curl may be imparted to the fibers by methods including chemical treatment or mechanical twisting. Curl is typically imparted before crosslinking or stiffening. Pulps may be stiffened by the use of crosslinking agents such as formaldehyde or its derivatives, glutaraldehyde, epichlorohydrin, methylolated compounds such as urea or urea derivatives, dialdehydes such as maleic anhydride, non-methylolated urea derivatives, citric acid or other polycarboxylic acids. Some of these agents are less preferable than others due to environmental and health concerns. Pulp may also be stiffened by the use of heat or caustic treatments such as mercerization. Examples of these types of fibers include NHB416 which is a chemically crosslinked southern softwood pulp fibers which enhances wet modulus, available from the Weyerhaeuser Corporation of Tacoma, Wash. Other useful pulps are debonded pulp (NF405) and non-debonded pulp (NB416) also from Weyerhaeuser. HPZ3 from Buckeye Technologies, Inc of Memphis, Tenn., has a chemical treatment that sets in a curl and twist, in addition to imparting added dry and wet stiffness and resilience to the fiber. Another suitable pulp is Buckeye HP2 pulp and still another is IP Supersoft from International Paper Corporation. Suitable rayon fibers are 1.5 denier Merge 18453 fibers from Acordis Cellulose Fibers Incorporated of Axis, Ala.

Hydroknit® material: Hydroknit® material is available from Kimberly-Clark Corporation of Dallas, Tex., USA and is a hydroentangled web of soft absorbent cellulosic fibers and spunbond synthetic fibers. The synthetic fibers are commonly polypropylene. The materials tested herein had a basis weight of 64 gsm and consists of only one ply of 75 weight percent pulp and 25 weight percent polypropylene spunbond fibers. As used herein the term “Hydroknit® with PP fibers” refers to the above described Hydroknit® fabric having an additional layer of spunbond polypropylene fibers deposited on its surface. This results in a coarse texture PP fiber layer on the Hydroknit® substrate to increase abrasion properties.

The term “spunbonded fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a collecting surface. Spunbond fibers are generally continuous and have average diameters (from a sample of at least 10) larger than 7 microns, more particularly, between about 10 and 20 microns.

Viva® Scrub Cloth: This material is a cellulosic paper towel and is available from Kimberly-Clark Corporation. It has a printed polyethylene acetate binder on both sides of the base-sheet which is composed of 72 weight percent softwood bleached kraft, 13 weight percent polyethylene vinyl acetate binder, 11 weight percent synthetic (polyester) fiber, 3 weight percent hardwood kraft, 1 weight percent total nitrogen.

Wypall® X80 material: Wypall® materials are also available from Kimberly-Clark Corporation. Wypall® X80 material is a highly absorbent, bulky Hydroknit® material having high wet strength and capacity. The materials tested herein had a basis weight of 125 gsm and were made from 75 weight percent pulp and 25 weight percent polypropylene spunbond fibers.

Airlaid fabric: “Airlaying” is a well-known airforming process by which a fibrous nonwoven layer can be formed. In the airlaying process, bundles of small fibers having typical lengths ranging from about 3 to about 52 millimeters (mm) are separated and entrained in an air supply and then deposited onto a forming screen, usually with the assistance of a vacuum supply. The randomly deposited fibers then are bonded to one another using, for example, hot air or a spray adhesive. The production of airlaid nonwoven composites is well defined in the literature and documented in the art. Examples include the DanWeb process as described in U.S. Pat. No. 4,640,810 to Laursen et al. and assigned to Scan Web of North America Inc, the Kroyer process as described in U.S. Pat. No. 4,494,278 to Kroyer et al. and U.S. Pat. No. 5,527,171 to Soerensen assigned to Niro Separation a/s, the method of U.S. Pat. No. 4,375,448 to Appel et al assigned to Kimberly-Clark Corporation, or other similar methods. The materials tested herein were made from 83 weight percent Weyerhaeuser CF405 pulp, and 17 weight percent latex binder (National Starch Dur-O-Set Elite PE) and had a basis weight of 68 gsm.

A number of other processes and materials may be used in the practice of the invention but not all were tested herein. Some of these other materials and processes are described below.

Point un-bonded or “PUB”, means a fabric pattern having continuous bonded areas defining a plurality of discrete un-bonded areas as illustrated in U.S. Pat. No. 5,858,515 to Stokes et al. The fibers or filaments within the discrete un-bonded areas are dimensionally stabilized by the continuous bonded areas that encircle or surround each un-bonded area, such that no support or backing layer of film or adhesive is required. The un-bonded areas are specifically designed to afford spaces between fibers or filaments within the un-bonded areas. A suitable process for forming point un-bonded nonwoven material includes providing a nonwoven fabric or web, providing opposedly positioned first and second calender rolls and defining a nip therebetween, with at least one of the rolls being heated and having a bonding pattern on its outermost surface comprising a continuous pattern of land areas defining a plurality of discrete openings, apertures or holes, and passing the nonwoven fabric or web within the nip formed by the rolls. Each of the openings in the roll or rolls defined by the continuous land areas forms a discrete un-bonded area in at least one surface of the nonwoven fabric or web in which the fibers or filaments of the web are substantially or completely un-bonded. Stated alternatively, the continuous pattern of land areas in the roll or rolls forms a continuous pattern of bonded areas that define a plurality of discrete un-bonded areas on at least one surface of the nonwoven fabric or web. Alternative embodiments of the aforesaid process includes pre-bonding the nonwoven fabric or web before passing the fabric or web within the nip formed by the calender rolls, or providing multiple nonwoven webs to form a pattern-un-bonded laminate.

The zero strain stretch process generally refers to a process in which at least two layers are bonded to one another while in an untensioned (hence zero strain) condition and where one of the layers is stretchable and elastomeric and the second is stretchable but not necessarily elastomeric. Such a laminate is stretched incrementally through the use of one or more pairs of meshing corrugated rolls which reduce the strain rate experienced by the web. This results in z-direction bulking of the laminate and subsequent elastic extensibility in the direction of initial stretching at least up to the point of initial stretching. Examples of such laminates and their production processes may be found in U.S. Pat. Nos. 5,143,679, 5,151,092, 5,167,897, and 5,196,000.

Z—directionally oriented fiber webs may also be used in the practice of this invention. A discussion of this process may be found in the October 1997 issue of Nonwovens Industry magazine at page 74 in an article by Krema, Jirsak, Hanus and Saunders entitled “What's New in Highloft Production?” as well as in Czech patents 235494 entitled “Fibre Layer, Method of its Production and Equipment for Application of Fibre Layer Production Method” issued May 15, 1995 and 263075 entitled “Method for Voluminous Bonded Textiles Production” issued Apr. 14, 1989.

Another suitable wipe includes those made according to U.S. Pat. No. 4,741,941, which teaches a nonwoven web with projections. The web from which the wipe will be made is formed onto a surface having projections with or without apertures and having a vacuum assist. The fabric has fibers with an array of hollow projections extending out of the fabric and separated by planar land areas. Fabric of this type may be made according to any of the nonwoven production techniques such as meltblowing, spunbonding, airlaying and the like.

Suitable wipes also include those taught in U.S. Pat. Nos. 4,775,582, 4,853,281 and 4,833,003. The '582 and '281 patents teach uniformly moist wipes made from polyolefin meltblown fibers. The '003 patent teaches uniformly moist wipes that have an abrasive surface bonded to a meltblown supporting layer.

A number of different treatments were used in the testing. The treatment of the webs was done as follows:

Kymene® 2064: A 0.1 weight percent Kymene® 2064 solution was prepared by diluting a stock Kymene® 2064 (from Hercules Inc., Wilmington, Del., USA) solution (20 weight percent solution in water, 5 mL) with de-ionized water (995 mL). Kymene® 2064 was “activated” by adjusting the solution pH with NaOH (0.4 M), which was measured at 8.8. Treatment of the substrates entailed a “dip and squeeze” protocol. Each substrate was submerged in the 0.1 weight percent Kymene® 2064 solution and agitated for approximately 1 minute to ensure saturation. The treated material was then squeezed to remove excess treatment solution using an Atlas Laboratory Wringer Type LW-1 (Atlas Electrical Devices Co., Chicago, Ill., USA) equipped with a 5 lb weight for the squeeze pressure. The material was cured at 100° C. for 20 minutes, allowed to cool to room temperature, and washed twice with de-ionized water. Excess water was removed using the same “dip and squeeze” protocol above. The washed material was allowed to dry at 100° C. for 30 minutes.

Kymene® 450: A 0.1 weight percent Kymene® 450 solution was prepared by diluting a stock Kymene® 450 (Hercules Inc.) solution (20 weight percent solution in water, 5 mL) with de-ionized water (995 mL). Kymene® 450 was “activated” by adjusting the solution pH with NaOH (0.4 M), which was measured at 9.2. Treatment of the substrates was performed in the same manner as with Kymene® 2064 above.

Kymene® 557 LX: A 0.1 weight percent Kymene® 557 LX solution was prepared by diluting a stock Kymene® 557 LX (Hercules Inc.) solution (12.5 weight percent solution in water, 8 mL) with de-ionized water (992 mL). The solution pH was adjusted with NaOH (0.4 M), which was measured at 8.0. Treatment of the substrates was performed in the same manner as with Kymene® 2064 above.

Kymene® 736: A 0.1 weight percent Kymene® 736 solution was prepared by diluting a stock Kymene® 736 (Hercules Inc., Wilmington, Del.) solution (38 weight percent solution in water, 2.6 mL) with de-ionized water (997.4 mL). The solution pH was adjusted with NaOH (0.4 M), which was measured at 8.0. Treatment of the substrates was performed in the same manner as with Kymene® 2064 above.

Alumina oligomer (aluminium chlorohydrol): A 1 weight percent alumina oligomer solution was prepared by diluting a stock alumina oligomer (from GEO Specialty Chemicals, Little Rock, Ark., USA) solution (50 weight percent solution in water, 20 mL) with de-ionized water (980 mL). The measured pH was 4.6. Treatment of the substrates entailed a “dip and squeeze” protocol. Each substrate was submerged in the 1 weight percent alumina oligomer solution and agitated for approximately 1 min to ensure saturation. The treated material was then squeezed to remove excess treatment solution using an Atlas Laboratory Wringer Type LW-1 (Atlas Electrical Devices Co.) equipped with a 5 lb weight for the squeeze pressure. The material was heated at 100° C. for 20 minutes, allowed to cool to room temperature, and washed twice with de-ionized water. Excess water was removed using the same “dip and squeeze” protocol above. The material was allowed to dry at 100° C. for 30 minutes.

Snowtex® AK nanoparticle (alumina-coated silica nanoparticles): A 1 weight percent Snowtex® AK nanoparticle solution was prepared by diluting a stock Snowtex® AK nanoparticle (from Nissan Chemicals Ltd, Houston, Tex., USA) solution (20 weight percent solution in water, 75 mL) with de-ionized water (1425 mL). The measured pH was 4.0. Treatment of the substrates entailed a “dip and squeeze” protocol. Each substrate was submerged in 1 weight percent Snowtex® AK nanoparticle solution and agitated for approximately 1 min to ensure saturation. The treatment solution for each substrate was not recycled for subsequent treatments. The treated material was then squeezed to remove excess treatment solution using an Atlas Laboratory Wringer Type LW-1 (Atlas Electrical Devices Co.) equipped with a 5 lb weight for the squeeze pressure. The material was heated at 100° C. for 20 minutes, allowed to cool to room temperature, and washed twice with de-ionized water. Excess water was removed using the same “dip and squeeze” protocol above. The material was allowed to dry at 100° C. for 30 minutes.

### Experiment 1

### Bacterial Growth Inhibition Test

The Kymene® class of chemicals are generally mild and not caustic to the skin. Certain of the Kymene® chemicals, however, are known to kill bacteria at some level. In order to determine whether the Kymene® treatment chemicals will escape from a wipe and perhaps kill the bacteria remaining on a surface, an assay was designed to measure the inhibition in bacterial cell growth of chemical leached from the treated materials. This assay procedure follows.

Two by two inch (5 by 5 cm) squares of treated and untreated materials were placed in 15 mL tubes containing 5 mL of sterile phosphate-buffered saline (PBS) solution. The tubes were placed in a shaking incubator at 37° C. for 2-3 hours. One milliliter of each solution was then transferred into a clean culture tube. Ampicillin-resistant E. Coli was added (10 microL, ˜1000 cells) to each tube. Sterile PBS was added to clean culture tubes as controls. The tubes were returned to the shaking incubator for 30 minutes more. One hundred microliters were removed from each tube and plated onto LB agar plates containing ampicillin. The plates were incubated at 37° C., and bacterial colonies were counted the following day to determine if there was Kymene® present in the solution that inhibited colony formation.

LB agar means Luria-Bertani broth (available from Difco and Becton Dickinson) in the amount of 25 grams mixed with agar (also from Difco and Becton Dickinson) in the amount of 15 grams and dissolved in 1 liter of distilled water and autoclaved. Circular plates (100 mm×15 mm) are poured after adding ampicillin (100 micrograms/mL) to the LB agar.

Data are listed in terms of the percent of colonies found on the plate compared to the PBS control.

| | | Percent of |
|---|---|---|
| Materials | Type of Kymene ® | Control |
| PBS Control | — | 100 |
| Spunlace | — | 100 |
| Spunlace | 2064 | 75 |
| Spunlace, softened | — | 87 |
| Spunlace, softened | 2064 | 82 |
| Fuzzy film, polyurethane | — | 84 |
| Fuzzy film, polyurethane | 2064 | 75 |
| Bonded Carded Web | — | 81 |
| Bonded Carded Web | 2064 | 100 |
| TCL | — | 80 |
| TCL | 2064 | 81 |
| TCL | 736 | 3 |
| Hydroknit ® | — | 93 |
| Hydroknit ® | 2064 | 81 |
| Hydroknit ® with PP | — | 69 |
| fibers | | |
| Hydroknit ® with PP | 2064 | 38 |
| fibers | | |
| Viva ® Scrub Cloth | — | 100 |
| Viva ® Scrub Cloth | 2064 | 95 |
| Wypall ® X80 | — | 100 |
| Wypall ® X80 | Aegis quaternary | 100 |
| | ammonium salt | |
| Wypall ® X80 | 2064 | 87 |
| Wypall ® X80 | 736 | 14 |

As can be seen from the results, none of the untreated materials except for the Hydroknit® with polypropylene (PP) fibers appeared to have had a dramatic effect on colony growth. The Kymene® 2064 materials did not show appreciable inhibition of growth except for the Hydroknit® with PP fibers. Materials treated with Kymene® 736 leached Kymene® into the solution, which killed most of the E. Coli in solution. This result is not surprising since it is known that Kymene® 2064 will cross-link to the above listed materials while Kymene® 736 generally will not. Treatments that are cross-linked to the substrate are more stable and less susceptible to leaching than are un-cross-linked treatments and are therefore desirable. A successful treatment will have at least 80 percent of the control sample cell colony growth, i.e., it will inhibit growth of 20 percent or less of the bacterial colonies.

### Experiment 2

### Bacterial Growth Inhibition Test

Snowtex® nanoparticles and aluminum oligomer were tested directly on E. Coli. Serial dilutions of both Snowtex® nanoparticles as well as the aluminum oligomer used to coat the nanoparticles were made in sterile PBS. One milliliter of each solution was added to a clean culture tube in duplicate. Sterile PBS was added to culture tubes as a control. Ampicillin-resistant E. Coli was added (10 microL, ˜1000 cells) to each solution. The culture tubes were placed in the 37° C. shaking incubator for 30 minutes. After the incubation, one hundred microliters were removed from each tube and plated onto LB agar plates containing ampicillin. Plates were incubated at 37° C., and bacterial colonies were counted the following day to determine if Snowtex® nanoparticles or the aluminum oligomer inhibited colony formation. Data are listed in terms of the percent of colonies found on the plate compared to the PBS control.

| Snowtex ® AK | Percent of | | Percent of |
|---|---|---|---|
| nanoparticle | Control | Al Oligomer | Control |
| PBS control | 100 | PBS Control | 100 |
| 10 mg/mL | 96 | 1% | 10 |
| 1 mg/mL | 99 | 0.5% | 57 |
| 0.5 mg/mL | 94 | 0.1% | 100 |
| 0.1 mg/mL | 100 | 0.05% | 100 |
| 0.05 mg/mL | 93 | 0.01% | 100 |
| 0.01 mg/mL | 89 | — | — |

The results show that Snowtex® nanoparticle nanoparticles did not have an effect on E. Coli colony formation, even at high concentrations (10 mg/mL). The aluminum oligomer decreased the number of E. Coli colonies when at concentrations 0.5% and higher. The concentration of the aluminum oligomer used to treat the nanoparticles is 1%. These results indicate that bacteria cell death will be observed only if all of the oligomer used to treat the materials leaches into the solution. As mentioned above, a successful treatment will have at least 80 percent of the control sample cell colony growth, i.e., it will kill 20 percent or less of the bacterial colonies.

### Experiment 3

### Method of Testing the Efficiency of Binding Bacteria

Not only must the successful treatment not kill substantial numbers of bacteria, it must also bind a large proportion of bacteria. In order to determine how efficient the wipe and treatment were in holding bacteria cleaned from the surface, the following test procedure was carried out.

Two by two inch (5 by 5 cm) squares of materials were cut and weighed in duplicate. Serial dilutions of an ampicillin-resistant E. Coli solution were made to achieve a final concentration of ˜10⁵ cells per mL. One hundred microliters of sterile PBS were added to each material. After 5 minutes, one hundred microliters of the bacteria solution were added onto each material. The materials were removed and placed into 10 mL of sterile PBS in 50 mL tubes. The tubes were sonicated (5 cycles of 30 seconds on, 30 seconds off) in a water bath to dislodge any bacteria that is not bound tightly to the material. One hundred microliters of the PBS solution from the tubes containing the material were plated in duplicate onto LB agar plates containing ampicillin. The plates were incubated at 37° C. and bacterial colonies were counted the following day. Data is shown as the percentage of reduction of bacteria in solution as compared to the PBS control.

| | | Reduction of |
|---|---|---|
| | | Bacteria in |
| | | Solution (%) |
| Material | Treatment | N = 4 |
| PBS control | — | 0 |
| Wypall ® X80 | — | 62 |
| Wypall ® X80 | Kymene ® 2064 | 72 |
| Wypall ® X80 | Kymene ® 450 | 63 |
| Wypall ® X80 | Kymene ® 557 | 40 |
| Wypall ® X80 | Al oligomer | 84 |
| Wypall ® X80 | Snowtex ® AK | 84 |
| | nanoparticle | |
| TCL | — | 47 |
| TCL | Kymene ® 2064 | 77 |
| TCL | Kymene ® 450 | 70 |
| TCL | Kymene ® 557 | 41 |
| TCL* | Al oligomer | 94, 97 |
| TCL | Snowtex ® AK | 95 |
| | nanoparticle | |
| Viva ® Scrub | — | 58, 48 |
| Cloth* | | |
| Viva ® Scrub | Kymene ® 2064 | 83, 73, 78 |
| Cloth* | | |
| Viva ® Scrub Cloth | Kymene ® 450 | 59 |
| Viva ® Scrub Cloth | Kymene ® 557 | 80 |
| Viva ® Scrub Cloth | Al oligomer | 82 |
| Viva ® Scrub Cloth | Snowtex ® AK | 68 |
| | nanoparticle | |
| Airlaid | — | 10 |
| Airlaid | Kymene ® 2064 | 64 |
| Airlaid | Kymene ® 450 | 67 |
| Airlaid | Kymene ® 557 | 36 |
| Airlaid | Al oligomer | 75 |
| Airlaid | Snowtex ® AK | 57 |
| | nanoparticle | |

*Materials tested multiple times

All materials, treated and untreated, showed a reduction in bacteria in the PBS solution after sonication. The most dramatic results are found in materials treated with Kymene® 2064, Kymene® 450, the aluminum oligomer, and in some cases, the Snowtex® AK nanoparticle nanoparticles. In all cases except for the Wypall® material, the treated materials showed a larger reduction in bacteria in solution than the untreated materials. It is desirable that the treated materials reduce bacterial growth according to this bacteria binding procedure by at least 50 percent, more desirably by at least 75 percent and still more desirably by at lease 90 percent.

### Experiment 4

### Streaming Zeta Potential Analysis—Used to Measure the Surface Charge of Treated Substrates

When an electrolyte solution is forced through a porous plug of material, a streaming potential develops due to the motion of ions in the diffusion layer which can be measured by an Electro Kinetic Analyzer (from Brookhaven Instruments Corporation, Holtsville, N.Y., USA). This value is then used to calculate the zeta potential according to the formula published by D. Fairhurst and V. Ribitsch (Particle Size Distribution II, Assessment and Characterization, Chapter 22, ACS Symposium Series 472, Edited by Provder, Theodore, ISBN 0841221170).

During the sample preparation, treated and untreated wipe substrates were cut to two identical pieces (120 mm×50 mm) and then placed into the sample cell with Teflon® spacers between them. After the sample cell was mounted onto the instrument, all the air bubbles were removed by purging. Then KCl solution (1 mM, pH=5.9, Temp=22° C.) was forced through the two layers of the media and Ag/AgCl electrodes were used to measure the streaming potential. All samples were tested under similar pH, solution conductivity and using the same number of spacers. Each testing was repeated 4 times, and the results are summarized in below table.

| | | Streaming Zeta |
|---|---|---|
| Material | Treatment | Potential (mV) |
| Wypall X80 | — | −1 |
| Wypall X80 | Kymene 2064 | +11 |
| Wypall X80 | Kymene 450 | +12 |
| Wypall X80 | Kymene 557 | +5 |
| Wypall X80 | Al oligomer | +8 |
| Wypall X80 | Snowtex AK | +25 |
| TCL | — | −2 |
| TCL | Kymene 2064 | +29 |
| TCL | Kymene 450 | +33 |
| TCL | Kymene 557 | +15 |
| TCL* | Al oligomer | +25 |
| TCL | Snowtex AK | +27 |
| Viva Scrub Cloth* | — | −11 |
| Viva Scrub Cloth* | Kymene 2064 | +23 |
| Viva Scrub Cloth | Kymene 450 | +22 |
| Viva Scrub Cloth | Kymene 557 | +11 |
| Viva Scrub Cloth | Al oligomer | +7.3 |
| Viva Scrub Cloth | Snowtex AK | +13 |
| Airlaid | — | −6 |
| Airlaid | Kymene 2064 | +40 |
| Airlaid | Kymene 450 | +38 |
| Airlaid | Kymene 557 | +31 |
| Airlaid | Al oligomer | +17 |
| Airlaid | Snowtex AK | +25 |

As can be seen from the data, the zeta potential for untreated substrates was negative, ranging from −11 mV to −1 mV at pH ˜5.9. The negative values for the untreated substrates indicate there should be repulsion between most bacteria and the untreated substrates. After treatment, the zeta potential for all the substrates became positive. The most cationically charged substrates are found to be materials treated with Kymene 2064, Kymene 450, the aluminum oligomer, and Snowtex AK nanoparticles.

As will be appreciated by those skilled in the art, changes and variations to the invention are considered to be within the ability of those skilled in the art. Examples of such changes are contained in the patents identified above, each of which is incorporated herein by reference in its entirety to the extent it is consistent with this specification. Such changes and variations are intended by the inventors to be within the scope of the invention. It is also to be understood that the scope of the present invention is not to be interpreted as limited to the specific embodiments disclosed herein, but only in accordance with the appended claims when read in light of the foregoing disclosure.

1. A wipe for the removal of bacteria comprising a nonwoven wipe having thereon a positively charged chemical.

2. The wipe of claim 1 where the positively charged chemical is embedded in the melt-extruded polymer fibers.

3. The wipe of claim 1 where the positively charged chemical is treated on the web.

4. The wipe of claim 1 wherein said chemical is a cationic polymer;

5. The wipe of claim 1 wherein said chemical is an epichlorohydrin-functionalized polyamine.

6. The wipe of claim 1 wherein said chemical is positively charged nanoparticles;

7. The wipe of claim 1 wherein said chemical is alumina oligomer;

8. The wipe of claim 3 where said wipe is treated with heat at a temperature and for a time sufficient to adhere a coating to the wipe.

9. The wipe of claim 1 wherein the wipe is made from a nonwoven fabric made according to a method selected from the group consisting of meltblowing, coforming, spunbonding, airlaying, bonding and carding, zero strain stretching and Z-directional orienting.

10. The wipe of claim 3 wherein said chemical is applied to said web in an amount between 0.01 and 10 weight percent on an aqueous basis.

11. The wipe of claim 3 wherein said chemical is applied to said web in an amount between 0.05 and 7 weight percent.

12. The wipe of claim 3 wherein said chemical is applied to said web in an amount between 0.1 and 5 weight percent on a aqueous basis.

13. The wipe of claim 4 wherein said treated wipe has a cell colony growth amount of at least 80 percent of a control cell colony growth amount.

14. The wipe of claim 1 wherein the treated wipe reduces bacterial growth according to a bacteria binding procedure by at least 50 percent.

15. The wipe of claim 1 wherein the treated wipe reduces bacterial growth according to a bacteria binding procedure by at least 75 percent.

16. The wipe of claim 1 wherein the treated wipe reduces bacterial growth according to a bacteria binding procedure by at least 90 percent.

17. A wipe for the removal of bacteria from surfaces comprising a cationic chemical coated onto a web and treatment of said coated wipe with heat at a temperature and for a time sufficient to adhere said chemical to said web.

18. The wipe of claim 17 comprising hydroentangled pulp and synthetic fibers.

19. The wipe of claim 17 comprising a bonding pattern selected from the group consisting of point unbonding, S-weave and creping.

20. A wipe for the removal of bacteria from surfaces comprising a web of pulp and synthetic fibers having thereon a cationic nanoparticle treatment.

21. The wipe of claim 20 made by a process selected from the group consisting of hydroentangling and coforming.

22. The wipe of claim 20 having a bonding pattern selected from the group consisting of point unbending, S-weave and creping.

23. A method of making a wipe to remove bacteria from a surface comprising the steps of providing a wipe, dipping said wipe in an aqueous solution of a non-antimicrobial treatment having a positive charge, squeezing said wipe to remove excess solution, and treating the wipe with heat at a temperature and for a time sufficient to crosslink said non-antimicrobial treatment.

24. The method of claim 23 where said non-antimicrobial treatment comprises a functionalized cationic polymer.
